(19) United States
(12) Patent Application Publication
(10) Pub. No.: US 2012/0083529 A1
(43) Pub. Date: Apr. 5, 2012

(54) USE OF JASMONATE ESTER DERIVATIVES FOR TREATING BENIGN HYPERPROLIFERATIVE SKIN DISORDERS

(75) Inventors: Max Herzberg, Sitrya (IL); Frederic Revah, Paris (FR)

(73) Assignee: SEPAL PHARMA LTD, Sitrya (IL)

(21) Appl. No.: 13/376,917

(22) PCT Filed: Jun. 3, 2010
(86) PCT No.: PCT/IL10/00438

§ 371 Date: Dec. 8, 2011

Related U.S. Application Data
(60) Provisional application No. 61/249,265, filed on Oct. 7, 2009.
(60) Provisional application No. 61/185,221, filed on Jun. 9, 2009.

(51) Int. Cl. A61K 31/215; A61P 17/00; G01N 33/53; C12Q 1/02; C12Q 1/68

(52) U.S. Cl. 514/530; 435/29; 435/6.12; 435/7.1

(57) ABSTRACT

The present invention relates to methods of treating benign hyperproliferative diseases of the epidermis by administering a composition comprising at least one jasmonate ester derivative, preferably methyl jasmonate. In particular, the present invention provides jasmonate ester derivatives as potent compounds useful for the treatment of disorders such as actinic keratoses with reduced side effects.

### FIELD OF THE INVENTION

The present invention relates to methods of use of jasmonate ester derivatives, e.g., methyl jasmonate for treating benign hyperproliferative disorders of the skin, in particular, actinic keratosis.

### BACKGROUND OF THE INVENTION

Jasmonates are a family of plant stress hormones, which are released in instances of extreme UV radiation, osmotic shock, heat shock, pathogen attack and the like, to initiate various cascades. The use of jasmonates for the treatment of mammalian cancer has been disclosed in International Patent Application WO 02/080890 and in U.S. Pat. No. 6,469,061 wherein the jasmonates were shown to induce direct cytotoxicity for various types of human cancer cells derived from breast, prostate, skin, and blood cancers. Methyl jasmonate was shown to be effective in preventing development of lymphomas in mice (Fingrut and Flescher, Leukemia, 16: 608-616, 2002).

International Patent Application WO 2005/054172 discloses halogenated jasmonate derivatives, pharmaceutical compositions comprising the derivatives, and their use in reducing cancer cell growth and in treating cancer.

International Patent Applications WO 2007/066336 and WO 2007/066337 disclose jasmonate derivatives, pharmaceutical compositions comprising same, and use thereof in reducing cancer cell growth and the treatment of cancer.

International Patent Application WO 2008/111088 discloses an assay for identifying anti-cancer candidate drug molecules by comparing the activity of the candidate drug molecule with the activity of a jasmonate derivative known as having anti-cancer effect in at least one of the following: dissociating hexokinase from mitochondria, interfering with hexokinase binding to a voltage dependent anion channel, and binding to hexokinase directly.

Kniazhanski et al. (Cancer Letters, 271(1): 34-46, 2008) discloses that methyl jasmonate is cytotoxic to a range of cervical cancer cell lines. Reischer et al. (Br J Pharmacol., 150(6): 738-749, 2007) discloses that methyl jasmonate suppresses cell motility and inhibits the development of lung metastases in metastatic melanoma cells.

Wang et al. (Society for Investigative Dermatology, 86^{th} annual meeting: abstract ID 861, 2007) discloses that jasmonic acid and methyl jasmonate are potential agents against UVB-induced skin cancer but have low toxicity on the malignant keratinocytes A431 cell line.

US Patent Application, Publication No. US 2003/0224024 discloses compositions of jasmonate esters, including methyl jasmonate, in the form of any cosmetic composition, including compositions for treating certain diseases of the skin, such as psoriasis. US Patent Application, Publication No. US 2010/0069497 discloses use of hydroxy jasmonate derivatives for treating psoriasis.

US Patent Application, Publication No. US 2009/0197939 discloses topical use of aromatic skin active ingredients, including methyl dihydro jasmonate for cosmetic applications, including treating skin disorders, such as seborrheic dermatitis, keratosis, psoriasis.

US Patent Application, Publication No. US 2007/0082852 relates to use of jasmonic acid for inducing proliferation of fibroblasts or keratinocytes thereby formation of new skin and gum tissues, facilitate wound healing, and ameliorate the effects of aging. It is explained that signs of aging may result from processes that include keratoses.

Skin benign hyperproliferative disorders arise from abnormal growth and differentiation of epidermal cells and may be attributed to lack of response or inappropriate response to regulating factors, or alternatively to dysfunctional regulating factors. This abnormality may develop into various benign skin disorders including, ichthyiosis, seborrhea and actinic keratoses.

Keratosis is defined as any horny growth of the skin including such growths as a wart or callous. Actinic keratosis typically is a sharply outlined verrucous or keratotic growth which may become malignant. It usually occurs in the middle aged or the elderly and is due to excessive exposure to the sun.

Actinic keratoses are potentially premalignant flat keratotic lesions considered to be either carcinoma in-situ or squamous intraepidermal neoplasia. Actinic keratoses are usually induced by ultraviolet (UV) radiation, typically from sunlight and are considered to be the most important manifestation of sun-induced skin damage. Actinic keratoses are characterized by alteration of maturation of keratinocytes from the basal layer of stratum corneum as viewed in microscopic examinations. The basal cells are enlarged, the nuclei are pleomorphic and some nuclei have nucleoli. These atypical cells replace part of or the entire thickness of epidermis (Histology: from normal microanatomy to pathology, Amenta et al. (Eds.), 7^{th} Edition, PICCIN, 1997). Untreated actinic keratoses may develop into basal cell carcinoma or squamous cell carcinoma.

Traditional treatments of actinic keratoses include the use of nonsteroidal anti-inflammatory drugs (e.g. diclofenac), immune response modifiers (e.g. imiquimod), cryosurgery, photodynamic therapy, electrocautery and chemotherapy agents, all of which are accompanied by undesirable side effects.

Hence, there is an unmet need for more potent compounds useful for treating benign hyperproliferative skin disorders with reduced side effects.

### SUMMARY OF THE INVENTION

The present invention is directed to methods of use of methyl jasmonate (MJ) and other jasmonate ester derivatives for treating benign hyperproliferative skin disorders.

The present invention is based in part on the unexpected finding that MJ exhibits cytotoxic activity towards certain keratinocyte cell lines. Nowhere in the background art is it taught or suggested that MJ or related jasmonate ester derivatives may be highly effective in treating benign hyperproliferative skin disorders including actinic keratoses. Furthermore, it is now disclosed for the first time that MJ can accumulate in the basal layer of the epidermis thus leading to high concentrations of the active ingredient upon topical administration. Surprisingly, such high concentrations in the epidermis were observed with MJ and related jasmonate ester derivatives, when applied topically. Moreover, these high epidermal concentrations were shown, for the first time, to induce inhibition of proliferation of abnormal benign epidermal cells. The present invention thus provides the use of MJ and other jasmonate ester derivatives as highly potent agents for treating benign hyperproliferative skin disorders with low levels of side effects.

According to one aspect, the present invention provides a method of treating a benign hyperproliferative skin disorder in a subject comprising administering to the subject an effective amount of a composition comprising at least one jasmonate ester derivative, other than methyl dihydro jasmonate, wherein the benign hyperproliferative skin disorder is not psoriasis. Preferably, the jasmonate derivative is Mi.

According to another aspect, the present invention provides a composition comprising an effective amount of at least one jasmonate ester derivative for treating a benign hyperproliferative skin disorder, wherein the benign hyperproliferative skin disorder is not psoriasis and the jasmonate ester derivative is not methyl dihydro jasmonate.

In yet another aspect, the present invention provides the use of an effective amount of at least one jasmonate ester derivative, for the preparation of a medicament for treating a benign hyperproliferative skin disorder.

In one currently preferred embodiment, the jasmonate ester derivative is methyl jasmonate.

Exemplary jasmonate ester derivatives include, but are not limited to a compound represented by formulae A, B or C:

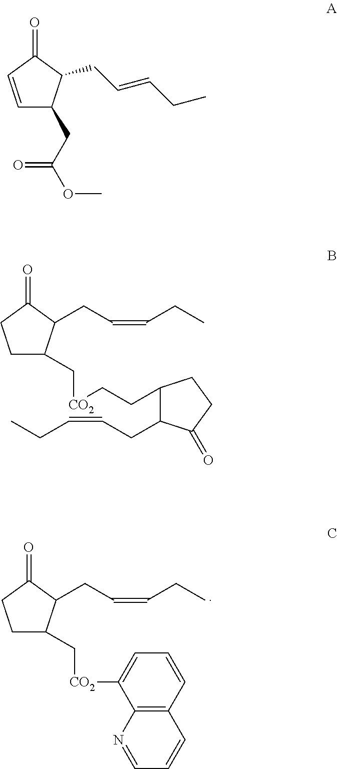

In certain embodiments, the jasmonate ester derivative is selected from the group consisting of 6-epi-cucurbic-acid-lactone, 12-hydroxy-jasmonic-acid-lactone, methyl-dihydro-isojasmonate, tuberonic acid-O-β-glucopyranoside, cucurbic acid-O-β-glucopyranoside and the lower alkyl esters of jasmonic acids such as jasmonic acid, 7-iso-jasmonic acid, 9,10-dihydrojasmonic acid, 2,3-didehydrojasmonic acid, 3,4-didehydrojasmonic acid, 3,7-didehydrojasmonic acid, 4,5-didehydrojasmonic acid, 4,5-didehydro-7-iso-jasmonic acid, cucurbic acid, 6-epi-cucurbic acid, 12-hydroxy-jasmonic acid, 11-hydroxy-jasmonic acid, 8-hydroxy-jasmonic acid, homo-jasmonic acid, dihomo-jasmonic acid, 11-hydroxy-dihomo-jasmonic acid, 8-hydroxy-dihomo-jasmonic acid, tuberonic acid, 5,6-didehydrojasmonic acid, 6,7-didehydro-jasmonic acid and, 7,8-didehydrojasmonic acid. Each possibility represents a separate embodiment of the invention.

Other ester derivatives of jasmonate are disclosed in U.S. Pat. No. 6,469,061, PCT International Patent Application Publication Nos. WO 02/080890, WO 2005/054172, WO 2007/066336, and WO 2007/066337, the contents of which are incorporated by reference herein in their entirety as if fully set forth herein. Exemplary suitable compounds from these references are depicted herein as formulae (I) to (VII) and A to C, and their use to treat benign hyperproliferative skin disorders represents separate embodiments of the present invention.

In particular embodiments, the methods disclosed herein provide the use of a composition comprising at least one jasmonate ester derivative of the present invention, formulated for topical administration.

In one embodiment, the compositions disclosed herein comprise at least one pharmaceutically acceptable excipient, carrier and/or diluent. In another embodiment, the active ingredient is dissolved in any acceptable lipid carrier. In yet another embodiment, the composition is in the form selected from an ointment, a gel and a cream.

In yet another aspect, the present invention provides an assay for determining the therapeutic potential of a jasmonate ester derivative in benign hyperproliferative skin disorders, comprising:
(a) introducing a viable mammalian skin explant obtained from skin having a benign hyperproliferative lesion, into a mammalian-avian chimeric model system comprising:
a fertilized avian egg within an egg shell, wherein a portion of the egg shell is removed creating an aperture, wherein the skin explant is in contact with the chorioallantoic membrane (CAM) of the fertilized avian egg such that vasculature extends from said fertilized avian egg to said skin explant;
b) incubating said fertilized avian egg for a period of time to allow engraftment;
c) contacting at least a portion of said explant with at least one jasmonate ester derivative to; and
d) examining said explant for a beneficial effect of the jasmonate ester derivative on the skin pathology.

According to an alternative embodiment, exposing at least a portion of the explant to the at least one jasmonate ester derivative occurs prior to step (a).

According to some embodiments, the avian embryo is selected from the group consisting of: chick embryos (Gallus gallus), turkey embryos (Meleagris gallopavo) and duck embryos (Anas platyrhyncha). The full experimental details of this model using an avian embryo bearing an explant of skin are disclosed in WO 2006/001021.

According to another embodiment, the explant is obtained from human skin.

According to yet another embodiment, the mammalian-avian chimeric model system further comprises means for resealing the egg thereby segregating the explant from the environment outside of the egg shell. According to yet another embodiment, the assay further comprises segregating the explant from the environment outside of the egg shell prior to incubation for a period of time to allow engraftment. According to yet another embodiment, the assay further comprises abrading the CAM before placing the explant on the CAM.

According to yet another embodiment, the at least one jasmonate ester derivative is contacted with the explant by topical administration, subcutaneous administration, injection into the explant, injection into the explant vasculature or injection into the fertilized avian egg vasculature.

According to yet another embodiment, the explant-egg system which is examined in step (d) of the assay, is selected from the group consisting of: at least a portion of the engrafted explant; at least a portion of the fertilized egg; at least a portion of a hematopoeitic organ of the avian embryo; a sample of blood extracted from the explant vasculature; a sample of blood extracted from the fertilized egg vasculature; and a sample of waste extracted from the allantois of the fertilized egg.

According to yet another embodiment, the hematopoietic organ of the avian embryo is spleen, bone marrow or liver.

According to yet another embodiment, the explant is examined by methods such as histological techniques, immunocytochemical techniques, biochemical techniques, molecular techniques, flow cytometry or polymerase chain reaction (PCR). According to yet another embodiment, the examining step includes estimating cell proliferation rate, connective tissue synthesis, tissue elasticity, blood vessel formation, epidermal differentiation, skin inflammation or fat deposition.

In specific embodiments, the benign hyperproliferative skin disorders to be treated according to the principles of the present invention, are selected from the group consisting of actinic keratoses, common warts, keratoacanthoma, seborrhoic keratosis, seborrhea and ichthyosis. Each possibility represents a separate embodiment of the invention.

In particular embodiments, the actinic keratoses are selected from the group consisting of actinic keratosis, hypertrophic actinic keratosis, Bowenoid actinic keratosis, arsenical keratosis, hydrocarbon keratosis, thermal keratosis, radiation keratosis, chronic scar keratosis, viral keratosis, actinic cheilitis, Bowen's disease, erythroplaquia of queyrat, oral erythroplaquia, leukoplakia and intraepidermal epithelialoma. Each possibility represents a separate embodiment of the invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows the distribution of methyl jasmonate in skin samples.

FIG. 2 presents the percutaneous absorption of methyl jasmonate in human frozen (2A) and fresh (2B) skin samples.

### DETAILED DESCRIPTION OF THE INVENTION

The present invention relates to compositions comprising jasmonate ester derivatives and methods of use thereof in treating benign hyperproliferative skin disorders.

Traditional treatments of benign skin disorders such as actinic keratoses include the use of nonsteroidal anti-inflammatory drugs (e.g. diclofenac), immune response modifiers (e.g. imiquimod), cryosurgery, photodynamic therapy, and electrocautery. Alternatively, chemotherapy agents such as 5-fluorouracil, colchicine, vinblastine sulfate, cyclophosphamide, azathioprine, cyclocytidine, azacytidine, azaserine, cisplatin, cycloheximide, mechlorethamine, cycloleucine, cytarabine, decarbazine, dactinomycin, dichloromethotrexate, emetrine hydrochloride, etoposide, quanazole, hydroxyurea, idoxuridine, mercaptopurine, methotrexate, methylglyoxal bis(guanylhydrazone), metoprine, pyrimethamine, scopolamine hydrobromide, thioquanine, thiotepa, vincristine sulface, and cyclosporin A, can be used. The most common chemotherapy agent currently applied for treating benign skin disorders is 5-fluorouracil which exerts cytotoxicity to the cells by inducing inflammation of the lesion followed by cell death. However, this treatment is accompanied by harsh side effects, the most common of which include diarrhea, nausea and vomiting, mouth sores, photophobia, low blood counts and severe inflammation.

Other known treatments of benign hyperproliferative skin disorders are also accompanied by many undesirable side effects including skin irritation, scaring, inflammation, sores, crust, eczema, burning sensation, and increased sensitivity to sunlight. Thus, there is an unmet need for therapeutic modalities which exhibit potency in treating these benign hyperproliferative epidermal pathologies with reduced side effects.

The present invention provides a novel application of jasmonate ester derivatives for the treatment of benign hyperproliferative and premalignant disorders of the skin. The present invention overcomes the drawbacks of the background art by providing the use of an effective amount of a jasmonate ester derivative for treating benign hyperproliferative skin disorders without exerting substantial side effects such as irritation and corrosion of the skin.

The present invention is based on the surprising finding that methyl jasmonate accumulates in the epidermis hence resulting in a substantially high level of MJ concentration, particularly in the basal layer of the epidermis. Without being bound by any theory or mechanism of action, it is contemplated that the reason for MJ accumulation is its low penetration through the epidermis. The low levels of esterases or lipases in the skin may also contribute to the accumulation of MJ in the epidermis due to its reduced conversion to jasmonic acid. Since methyl jasmonate was shown to have improved stability in cutaneous penetration studies, the accumulation of MJ in the basal layer of the epidermis renders its use for treating benign hyperproliferative disorders of the skin, extremely advantageous.

### Jasmonate Derivatives

Any ester derivative of jasmonate can be used in the compositions of the present invention. As used herein, the term “ester derivative” includes the natural plant hormone methyl jasmonate, as well as any natural or synthetic ester derivative of jasmonic acid including all salts, hydrates, solvates, polymorphs, optical isomers, geometrical isomers, enantiomers, and diastereomers of the particular jasmonate ester derivative; and mixtures thereof.

In a currently preferred embodiment, the jasmonate ester derivative is methyl jasmonate, which is chemically designated as methyl 3-oxo-2-(2-pentenyl) cyclopentaneacetic acid.

In another currently preferred embodiment, the jasmonate ester derivative is a compound represented by formula A:

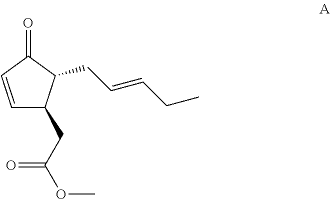

In another currently preferred embodiment, the jasmonate ester derivative is a compound represented by formula B:

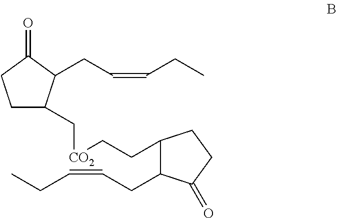

In another currently preferred embodiment, the jasmonate ester derivative is a compound of formula C.

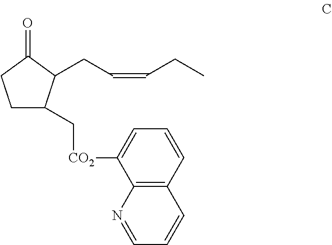

In other embodiments, the jasmonate ester derivative includes, but is not limited to, ester derivatives described in A) U.S. Pat. No. 6,469,061 and PCT International Patent Application Publication No. WO 02/080890; B) PCT International Patent Application Publication No. WO 2005/054172; C) PCT International Patent Application Publication No. WO2007/066336; D) PCT International Patent Application Publication No. WO2007/066337, and E) jasmonate-amino acid conjugate compounds, the contents of which are incorporated by reference herein in their entirety as if fully set forth herein. Non-limiting examples of such jasmonate derivatives include compounds represented by any of formula I through VII as set forth hereinbelow. Each possibility represents a separate embodiment of the present invention.
A) Compounds disclosed in U.S. Pat. No. 6,469,061 and WO 02/080890, represented by the structure of formula I:

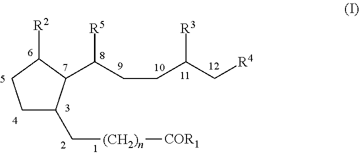

wherein:
n is 0, 1, or 2;
R¹ is alkoxy or O-glucosyl,
R² is OH, O, alkoxy or O-glucosyl,
R³, R⁴ and R⁵ are H, OH, alkoxy or O-glucosyl, and/or wherein R¹ and R², or R¹ and R⁴ together form a lactone,
the bonds between C3:C7, C4:C5, and C9:C10 may be double or single bonds, wherein at least one of the bonds between C3:C7, C4:C5, and C9:C10 is a double bond;
   and salts, hydrates, solvates, polymorphs, optical isomers, geometrical isomers, enantiomers, diastereomers, and mixtures thereof.

Exemplary jasmonate ester derivatives of formula (I) include, but are not limited to, methyl jasmonate, 6-epi-cucurbic-acid-lactone, 12-hydroxy-jasmonic-acid-lactone, methyl-dihydro-isojasmonate, tuberonic acid-O-β-glucopyranoside, cucurbic acid-O-β-glucopyranoside, and the lower alkyl esters of any of the following acids: jasmonic acid, 7-iso-jasmonic acid, 2,3-didehydrojasmonic acid, 3,4-didehydrojasmonic acid, 3,7-didehydrojasmonic acid, 4,5-didehydrojasmonic acid, 4,5-didehydro-7-iso-jasmonic acid, cucurbic acid, 6-epi-cucurbic acid, 12-hydroxy-jasmonic acid, 11-hydroxy-jasmonic acid, 8-hydroxy-jasmonic acid, homo-jasmonic acid, dihomo-jasmonic acid, 11-hydroxy-dihomo-jasmonic acid, 8-hydroxy-dihomo-jasmonic acid, tuberonic acid, 5,6-didehydrojasmonic acid, 6,7-didehydrojasmonic acid and, 7,8-didehydrojasmonic acid.
B) Compounds disclosed in WO 2005/054172, represented by the structure of formula II:

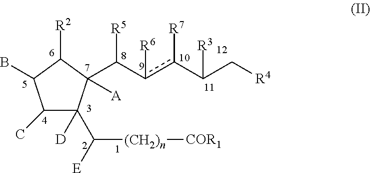

wherein
n is 0, 1, or 2;
R¹ is C₁ to C₁₂ alkoxy, C₁ to C₁₂ substituted alkoxy, aryloxy or O-glucosyl;
R² is OH, C₁ to C₁₂ alkoxy, C₁ to C₁₂ substituted alkoxy, O-glucosyl, oxo, alkyl or imino;
R³, R⁴, R⁵, R⁶, R⁷, A, B, C, D and E are each independently H, halogen, OH, C₁ to C₁₂ alkoxy, C₁ to C₁₂ substituted alkoxy, aryloxy, O-glucosyl, C₁ to C₁₂ alkyl or C, to C₁₂ substituted alkyl; wherein R¹ and R², or R¹ and R⁴ may form together a lactone which is optionally substituted; wherein the bonds between C3:C7, C4:C5, and C9:C10 may independently be double bonds or single bonds;
provided that at least one of R³, R⁴, R⁵, R⁶, R⁷, A, B, C, D and E is a halogen
   and salts, hydrates, solvates, polymorphs, optical isomers, geometrical isomers, enantiomers, diastereomers, and mixtures thereof.

Exemplary jasmonate ester derivatives of formula (II) include, but are not limited to: methyl jasmonate di-bromide (MJDB), methyl jasmonate tetrabromide (MJTB), a compound wherein R⁶ and R⁷ are each fluoro, a compound wherein R⁶ and R⁷ are each iodo, a compound wherein R⁶ and R⁷ are each chloro, a compound wherein one of R⁶ and R⁷ is iodo and the other is hydroxy, and a compound wherein one of R⁶ and R⁷ is iodo and the other is methoxy.
C) Compounds disclosed in WO 2007/066336, represented by the structure of formula III:

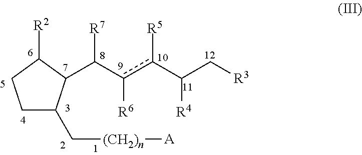

wherein
   A is COR¹;
R¹ is selected from the group consisting of
a) heteroaryloxy; and
b) —O[(CH₂)ₚO)]ₘ—R¹²;
R² is selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, OR⁸, oxo and NR^{9a}R^{9b};
R³, R⁴, R⁵, R⁶ and R⁷ are each independently selected from the group consisting of hydrogen, halogen, unsubstituted or substituted C₁-C₁₂ alkyl, unsubstituted or substituted C₁-C₁₂ haloalkyl, unsubstituted or substituted C₃-C₈ cycloalkyl, unsubstituted or substituted aryl, unsubstituted or substituted heteroaryl, OR⁸ and NR^{9a}R^{9b},
or R⁵ and R⁶ together with the carbons to which they are attached form a C₃-C₈ cycloalkyl or a C₃-C₈ cycloalkyl substituted by halo;
or one of R⁵ and R⁶ represents an oxygen atom which is bonded to C₆, thereby forming an oxygen-containing 6 or 5 membered heterocyclic ring, respectively;
wherein the bond between C₉ and C₁₀ can be a single or double bond;
R⁸, R^{9a} and R^{9b} are each independently selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, glucosyl, or R^{9a} and R^{9b} can together with the nitrogen to which they are attached form an unsubstituted or substituted heterocyclic or heteroaromatic ring optionally containing one or more additional heteroatom selected from O, N and S;
R¹² is a hydrogen or a hydroxy protecting group;
n is selected from 0, 1 and 2;
m is an integer of 1 to 20; and
p is an integer of 1 to 12;
including salts, hydrates, solvates, polymorphs, optical isomers, geometrical isomers, enantiomers, diastereomers, and mixtures thereof.

Specific examples of the compounds of formula III include, but are not limited to:

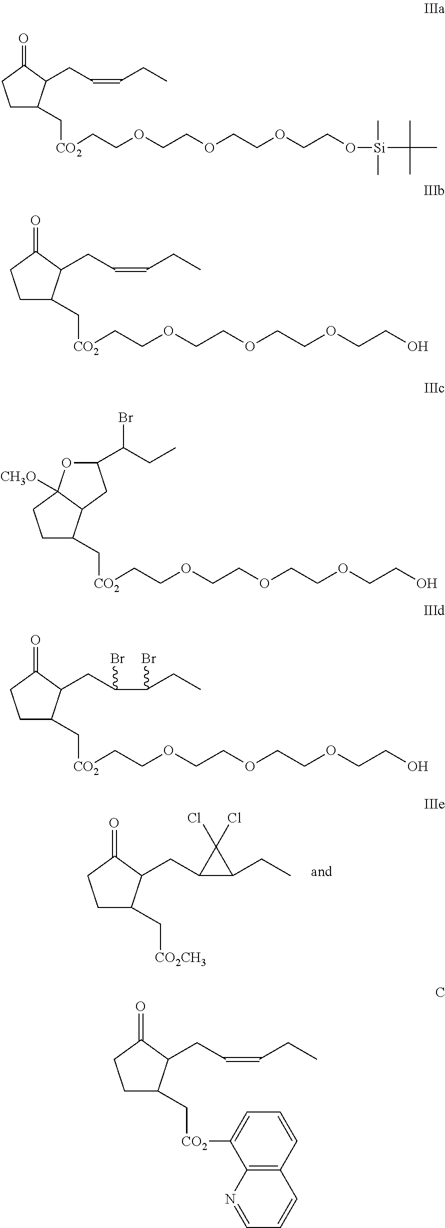

Another example includes a jasmonate derivative represented by the structure of formula IIIf.

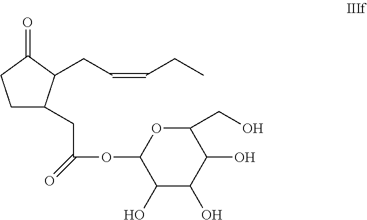

D) Compounds disclosed in WO 2007/066337, including:
   (i) Compounds represented by the structure of formula IV:

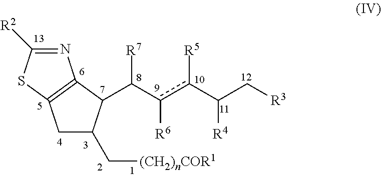

wherein
n is 0,1, or 2;
R¹ is OR⁸;
R² is selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, OR⁸, NR^{9a}R^{9b}, NHCOR¹⁰ and NHSO₂R¹¹;
R³, R⁴, R⁵, R⁶ and R⁷ are each independently selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, OR⁸ and NR^{9a}R^{9b};
wherein the bond between C₉ and C₁₀ can be a single or a double bond; and
R⁸, R^{9a}, R^{9b}, R¹⁰ and R¹¹, are each independently selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, glucosyl, or R^{9a} and R^{9b} can together with the nitrogen to which they are attached form an unsubstituted or substituted heterocyclic or heteroaromatic ring optionally containing one or more additional heteroatom selected from O, N and S;
including salts, hydrates, solvates, polymorphs, optical isomers, geometrical isomers, enantiomers, diastereomers, and mixtures thereof.

A non-limiting example of the compounds of formula IV is:

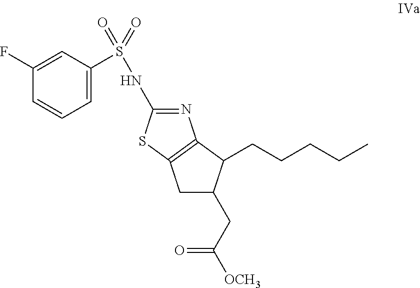

(ii) Compounds represented by the structure of formula V:

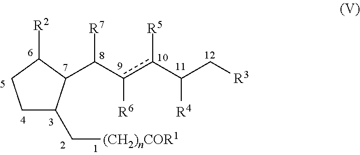

wherein
n is independently at each occurrence 0, 1, or 2;
R¹ is a group of the formula:

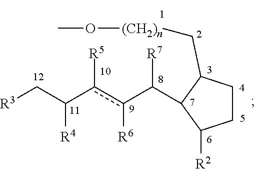

R² is independently at each occurrence selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, OR⁸, oxo and NR^{9a}R^{9b};
R³, R⁴, R⁵, R⁶ and R⁷ are each independently at each occurrence selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, OR⁸ and NR^{9a}R^{9b};
wherein the bond between C₉ and C₁₀ can independently at each occurrence be a single or a double bond; and
R⁸, R^{9a} and R^{9b} are each independently at each occurrence selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, glucosyl, or R^{9a} and R^{9b} can together with the nitrogen to which they are attached form an unsubstituted or substituted heterocyclic or heteroaromatic ring optionally containing one or more additional heteroatom selected from O, N and S;
including salts, hydrates, solvates, polymorphs, optical isomers, geometrical isomers, enantiomers, diastereomers, and mixtures thereof

A specific non-limiting example of the compounds of the formula V is represented by the structure of formula Va:

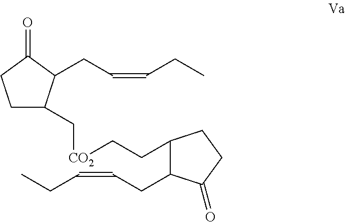

(iii) Dimeric, oligomeric or polymeric jasmonate derivatives comprising a plurality of covalently linked jasmonic acid moieties represented by the structure of formula VI:

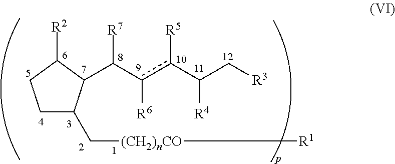

wherein
n is independently at each occurrence 0, 1, or 2;
p is 2, 3, 4, 5 or 6;
R¹ a linker selected from the group consisting of —O—, polyoxy and a sugar moiety;
R² is independently at each occurrence selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, OR⁸, oxo and NR^{9a}R^{9b};
R³, R⁴, R³, R⁶ and R⁷ are each independently at each occurrence selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, OR⁸ and NR^{9a}R^{9b};
wherein the bond between C₉ and C₁₀ can independently at each occurrence be a single or a double bond; and
R⁸, R^{9a} and R^{9b} are each independently at each occurrence selected from the group consisting of hydrogen, unsubstituted or substituted C₁-C₁₂ alkyl, unsubstituted or substituted C₃-C₈ cycloalkyl, unsubstituted or substituted aryl, unsubstituted or substituted heteroaryl, glucosyl, or R^{9a} and R^{9b} can together with the nitrogen to which they are attached form an unsubstituted or substituted heterocyclic or heteroaromatic ring optionally containing one or more additional heteroatom selected from O, N and S;

including salts, hydrates, solvates, polymorphs, optical isomers, geometrical isomers, enantiomers, diastereomers, and mixtures thereof.

Specific examples of the compounds of the formula VI include, but are not limited to:

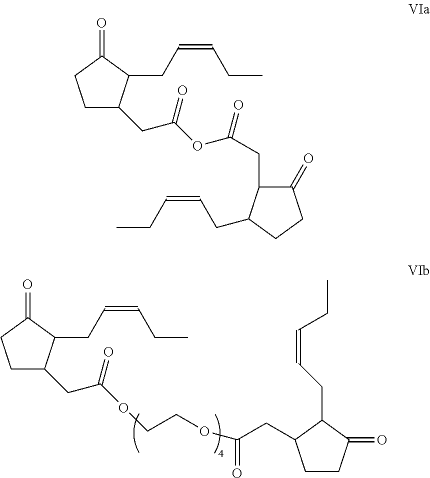

e) Oligomeric compounds comprising a plurality of jasmonate moieties linked via a linker sugar moiety, represented by the structure of formula VII:

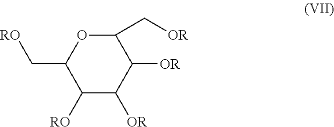

wherein
R is represented by the formula:

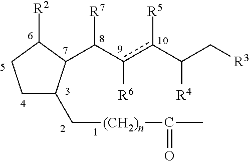

wherein each of R², R³, R⁴, R⁵, R⁶ and R⁷ is as defined above.

A specific example of the compounds of the formula VII is represented by the structure of formula VIIa:

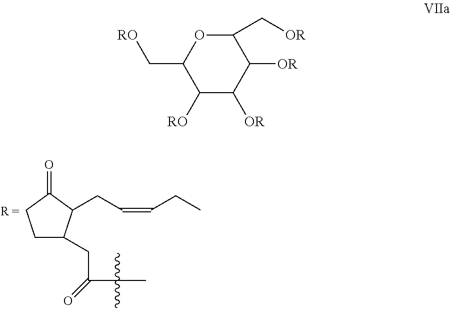

All stereoisomers of the jasmonate ester derivatives are contemplated, either in admixture or in pure or substantially pure form. The jasmonate derivatives can have asymmetric centers at any of the atoms. Consequently, the compounds can exist in enantiomeric or diastereomeric forms or in mixtures thereof. The present invention contemplates the use of any racemates (i.e. mixtures containing equal amounts of each enantiomers), enantiomerically enriched mixtures (i.e., mixtures enriched for one enantiomer), pure enantiomers or diastereomers, or any mixtures thereof. The chiral centers can be designated as R or S or R,S, D or d or L or l or d,l or D,L. In addition, several of the compounds of the invention contain one or more double bonds. The present invention intends to encompass all structural and geometrical isomers including cis, trans, E and Z isomers, independently at each occurrence.

One or more of the compounds of the invention, may be present as a salt. The term “salt” encompasses both basic and acid addition salts, including but not limited to, carboxylate salts or salts with amine nitrogens, and include salts formed with the organic and inorganic anions and cations discussed below. Furthermore, the term includes salts that form by standard acid-base reactions with basic groups (such as amino groups) and organic or inorganic acids. Such acids include hydrochloric, hydrofluoric, trifluoroacetic, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicylic, methanesulfonic, benzenesulfonic, sorbic, picric, benzoic, cinnamic, and like acids.

The term “organic or inorganic cation” refers to counter-ions for the carboxylate anion of a carboxylate salt. The counter-ions are chosen from the alkali and alkaline earth metals (such as lithium, sodium, potassium, barium, aluminum and calcium); ammonium and mono-, di- and tri-alkyl amines such as trimethylamine, cyclohexylamine; and the organic cations, such as dibenzylammonium, benzylammonium, 2-hydroxyethylammonium, bis(2-hydroxyethyl)ammonium, phenylethylbenzylammonium, dibenzylethylenediammonium, and like cations. See, for example, Berge et al., J. Pharm. Sci., 66: 1-19, 1977. Other cations encompassed by the above term include the protonated form of procaine, quinine and N-methylglucosamine, and the protonated forms of basic amino acids such as glycine, ornithine, histidine, phenylglycine, lysine and arginine. Furthermore, any zwitterionic form of the instant compounds formed by a carboxylic acid and an amino group is also contemplated.

The present invention also includes solvates of the compounds of the present invention and salts thereof “Solvate” means a physical association of a compound of the invention with one or more solvent molecules. This physical association involves varying degrees of ionic and covalent bonding, including hydrogen bonding. In certain instances the solvate will be capable of isolation. “Solvate” encompasses both solution-phase and isolatable solvates. Non-limiting examples of suitable solvates include ethanolates, methanolates and the like. “Hydrate” is a solvate wherein the solvent molecule is water.

The present invention also includes polymorphs of the compounds of the present invention and salts thereof. The term “polymorph” refers to a particular crystalline state of a substance, which can be characterized by particular physical properties such as X-ray diffraction, IR or Raman spectra, melting point, and the like.

### Pharmaceutical Compositions

Although the compounds of the invention can be administered alone, it is contemplated that they will be administered in pharmaceutical compositions further containing at least one pharmaceutically acceptable carrier, excipient or diluent.

The pharmaceutical compositions of the present invention are formulated for topical administration, e.g. as an ointment, a gel or a cream. For topical administration to body surfaces using, for example, creams, gels, drops, ointments and the like, the compounds of the present invention can be prepared and applied in a physiologically acceptable diluent with or without a pharmaceutical carrier. Adjuvants for topical or gel base forms may include, for example, sodium carboxymethylcellulose, polyacrylates, polyoxyethylene-polyoxypropylene-block polymers, polyethylene glycol and wood wax alcohols.

Such compositions are prepared in a manner well known in the pharmaceutical art and comprise as an active ingredient at least one compound of the present invention as described hereinabove, and a pharmaceutically acceptable carrier, excipient or diluent. The term “pharmaceutically acceptable” means approved by a regulatory agency of the Federal or a state government or listed in the U.S. Pharmacopeia or other generally recognized pharmacopeia for use in mammals and, more particularly, in humans.

During the preparation of the pharmaceutical compositions according to the present invention the active ingredient is usually mixed with a carrier or excipient, which may be liquid, semi-liquid material (e.g. gel) or semi-solid material. The topical compositions of the invention can be formulated into any medium acceptable for dermatological application. For example, the compositions can be formulated into solutions, creams, lotions, emulsions, suspensions and the like. Dermatologically acceptable excipients useful for the production of such formulations are well known to a skilled artisan and include, but are not limited to, semi-solid and liquid petroleum fractions. The petrolatum can be a synthetic or semi-synthetic hydrocarbon of the same nature as petrolatum. Mixtures of such ingredients can also be used. The preferred semi-solid material is petrolatum, commercially available from a wide variety of sources. The excipient, according to the principles of the present invention includes any synthetic or semi-synthetic oleaginous liquid fraction including, but not limited to mineral oil, and propylene glycol. Other suitable excipients include emulsifiers and thickeners selected from cetyl alcohol, stearyl alcohol, stearic acid, palmitic acid, and mixtures thereof.

Yet other suitable dermatologically acceptable excipients include thickeners which provide a high viscosity cream designed to local application to skin lesions. Exemplary thickeners include a mixture of a carbomer and triethanolamine. The mixture is combined together and added to the composition in an amount ranging from about 0.05 to 5 weight percent.

The formulations can additionally include lubricating agents such as talc, magnesium stearate, and mineral oil; wetting agents, surfactants, emulsifying and suspending agents; preserving agents such as methyl- and propyl-hydroxybenzoates; colorants, buffering agents (e.g., acetates, citrates or phosphates), disintegrating agents, moistening agents, antibacterial agents, chelating agents (e.g., ethylenediaminetetraacetic acid), and agents for the adjustment of tonicity such as sodium chloride.

In currently preferred embodiments, the pharmaceutical compositions of the present invention formulated for topical administration comprise one or more antioxidants. Suitable antioxidants include, but are not limited to, tocopherols (vitamin E), tocopherol derivatives, tocotrienols, ascorbic acid (vitamin C), ascorbic acid derivatives, sodium bisulfite, carotenoids, vitamin A or derivatives thereof, butylated hydroxytoluene, butylated hydroxyanisole, gallic esters, flavonoids such as, for example, quercetin or myricetin, selenium, grape seed extract, catechins such as, for example, epicatechin, epicatechingallate, epigallocatechin or epigallocatechingallate, sulfur-containing molecules such as, for example, glutathione, cysteine, lipoic acid, N-acetylcysteine, chelating agents such as, for example, ethylenediamine tetraacetic acid or other customary antioxidants. In one embodiment, antioxidants are present in a composition of the invention at about 0.1 to about 20 weight percent.

Suitable pharmaceutical carriers include, but are not limited to, sterile liquids, such as oils, including those of petroleum, animal, vegetable or synthetic origin, such as peanut oil, soybean oil, mineral oil, sesame oil and the like, polyethylene glycols, propylene glycol, glycerin, or other synthetic solvents.

The pharmaceutical compositions of the present invention may further comprise glyceryl stearate, which is a monoester of glycerine and stearic acid, or other suitable forms of glyceryl stearate (e.g. glyceryl stearate SE, which is a commercially available self-emulsifying grade of glycerol stearate that contains some sodium and/or potassium stearate). Glyceryl stearate may be in the composition anywhere from about 1 to about 3 weight percent.

According to the principles of the present invention, xanthan gum may be further added to the composition. Xanthan gum is a high molecular weight heteropolysaccharide gum produced by pure-culture fermentation of a carbohydrate with Xanthomonas campestris. The gum is also commercially available from various sources.

Another formulation employed in the methods of the present invention is transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art.

### Therapeutic Use

The present invention is directed to a method of treating benign hyperproliferative skin disorders by administering to a subject a pharmaceutical composition comprising at least on jasmonate ester derivative as described herein.

Further provided in the present invention is the use of at least one jasmonate ester derivative in the preparation of a medicament for treating benign hyperproliferative skin disorders.

There is also provided in the present invention a pharmaceutical composition comprising at least one jasmonate ester derivative as described above for the treatment of benign hyperproliferative skin disorders.

The methods according to the principles of the present invention are designated for mammals, in particular, humans.

The pharmaceutical compositions comprise an effective amount of at least one jasmonate ester derivative. The term “therapeutically effective amount” or “an effective amount” as used herein refers to a quantity of a compound which is sufficient to provide a beneficial effect to the subject to which the compound is administered. The effective amount, according to the principles of the present invention can be determined by any one of ordinary skill in the art and can be tested on various models both in vitro and in vivo. A therapeutically effective amount, according to the principles of the present invention refers to an amount which improves, in a measurable manner, the differentiation of the epidermal cells as determined for example by indirect immunofluorescence analysis. Alternatively the therapeutically effective amount is an amount which can decrease, to a measurable amount, the proliferation of the cells as indicated by measurement of the activity of mitochondrial dehydrogenase enzymes of living cells (MTT assay) and by counting of basal cells level.

The term “treating” as used herein refers to alleviation of the adverse effects of the disease or disorder, which alleviation may be manifested by a decrease in at least one of the following: reduction in the number of abnormal epidermal cells (due to cell death which may be necrotic, apoptotic or any other type of cell death or combinations thereof) as compared to control; decrease in proliferation of cells, i.e. the total number of cells may increase but at a lower level or at a lower rate than the increase in control; or decrease in the invasiveness of cells (as determined for example by soft agar assay) as compared to control even if their total number has not changed.

The abnormal epidermal cells, according to the principles of the present invention, are hyperproliferative benign cells, such as human keratinocytes from psoriatic skin, and precancerous keratinocytes. The keratinocytes may be from actinic keratoses, keratoacanthoma, common warts or seborrhoic keratoses lesions. The abnormal epidermal cells may also be from other benign skin disorders such as, but not limited to, ichthyosis.

The precancerous keratinocytes, according to the principles of the present invention, are atypical epidermal keratinocytes that are characterized by at least one of the following features: nuclear pleomorphism, hyperchromatism, loss of normal cellular polarity, premature keratinization (dyskeratosis), and increased number of mitotic figures.

The term “treating benign hyperproliferative skin disorders” in the context of the present invention includes at least one of the following: a decrease in the rate of growth of the lesions; or cessation of growth of the lesions characteristic of the skin disorder. In a currently preferred embodiment, the lesions which are characteristic of the hyperproliferative skin disorder are diminished, reduced in size or totally eliminated.

The benign hyperproliferative skin disorders according to the principles of the present invention include, but are not limited to, actinic keratoses, common warts, keratoacanthoma, seborrhoic keratosis, seborrhea and ichthyosis.

Actinic keratosis typically is a sharply outlined verrucous or keratotic growth which may become malignant. The term “actinic keratoses” as used in the context of the present invention includes precancerous skin lesions of keratinocytes which are areas of skin in which tissue shows the tendency to develop into cancer, although the tissue in its present state is not cancerous. Epithelial precancerous lesions include actinic keratosis (also called solar keratosis or senile keratosis), hypertrophic actinic keratosis, Bowenoid actinic keratosis, arsenical keratosis, hydrocarbon keratosis, thermal keratosis, radiation keratosis, chronic scar keratosis, viral keratosis, actinic cheilitis, Bowen's disease, erythroplaquia of queyrat, oral erythroplaquia, leukoplakia, and intraepidermal epithelialoma.

In a currently preferred embodiment, the jasmonate ester derivatives, according to the principles of the present invention, are used for the treatment of actinic keratosis. Actinic keratosis is usually present as lesions on the skin which may or may not be visually detectable exhibiting various sizes and shapes. Actinic keratosis is characterized by an inflammatory infiltration of lymphocytes, histocytes and a variable number of plasma cells. It is further characterized by the proliferation of keratinocytes.

Evaluating Therapeutic Efficiency and Determining Administration Regimen

In another embodiment, the present invention provides assays for determining the efficacy of jasmonate ester derivative in treating benign hyperproliferative skin disorders such as actinic keratoses. The assays provide a number of advantages. For instance, in various embodiments, the lack of an egg-shell, or near lack of an egg-shell allows for easier viewing and monitoring the embryo of the fertilized egg. Furthermore, in various embodiments there is greater access to the blood and allantoic waste of the fertilized egg, making it is possible to obtain blood or waste samples.

The assays of the invention are based on an animal model disclosed in WO 2006/001021, the contents of which are incorporated by reference herein in their entirety. In this model, chimeric avian embryos comprising mammalian skin grafts having a benign hyperproliferative skin serve as a convenient and efficient system for screening therapeutic treatments. Using the avian model, the various physiological and pathological processes occurring in response to treatment of the skin graft with the jasmonate ester derivatives of the invention, can be monitored. Furthermore, the assay of the invention allows examining the therapeutic effect of the tested jasmonate ester derivative on the skin graft thereby determining the therapy (administration) regimen for said jasmonate ester derivative on benign hyperproliferative skin disorder.

Although administration regimen can be determined by a skilled artisan depending on the condition and the severity of the lesions, the patient population, age, weight etc., applying the knowledge gained from the changes observed in the mammalian-avian chimeric model system, provide more accurate and powerful guidelines. The changes observed in the mammalian-avian chimeric model system upon treatment with at least one jasmonate ester derivative include, but not limited to, formation of connective tissue, inflammation and improved tissue elasticity of the skin graft. The magnitude of these changes is used to select preferred modes of administration and optimal dosage ranges. The assay may also be used to determine the therapeutic efficacy of the jasmonate ester derivative in combination with other therapeutic agent.

Thus, the compositions of the invention may be administered once-daily, twice-daily, thrice daily, once-weekly or once-monthly. In addition, the administration can be continuous, i.e., every day, or intermittently. The terms “intermittent” or “intermittently” as used herein means stopping and starting at either regular or irregular intervals. For example, intermittent administration can be administration one to six days per week or it may mean administration in cycles (e.g. daily administration for two to eight consecutive weeks, then a rest period with no administration for up to one week) or it may mean administration on alternate days.

Should the compositions of the present invention be administered as a combination therapy with additional therapeutic agents (e.g. inflammatory drugs, chemotherapy agents etc), the treatment may take place sequentially in any order, simultaneously or a combination thereof. For example, administration of a jasmonate ester derivative can take place prior to, after or at the same time as administration of the additional therapeutic agent(s). For example, a total treatment period can be decided for the jasmonate ester derivative. The additional agent(s) can be administered prior to onset of treatment with the jasmonate ester derivative or following treatment with the jasmonate ester derivative. In addition, the additional agent(s) can be administered during the period of jasmonate ester derivative administration but does not need to occur over the entire jasmonate ester derivative treatment period. In another embodiment, the treatment regimen includes pre-treatment with one agent, followed by the addition of the other agent or agents. Alternating sequences of administration are also contemplated. Alternating administration includes administration of a jasmonate ester derivative, followed by the additional agent, followed by a jasmonate ester derivative, etc.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

### Pharmacokinetics Studies in Human Skin

In order to test the applicability of jasmonate ester derivatives in treating benign hyperproliferative skin disorders through topical administration, samples of human skin were used to assess dermal drug delivery and percutaneous absorption of MJ. The penetration profile of MJ in human abdominal skin was analyzed using an in vitro flow-through diffusion Frantz cell, according to the OECD guidelines and ECVAM recommendations (OECD Guideline for the testing of chemicals, 428, Skin absorption: in vitro method, adopted Apr. 13, 2004; Hows, The report and recommendation of ECVAM workshop 13, ATLA, 24, 81, 1996). These studies were conducted by BSL-Bioservices (Planegg, Germany) and the samples were analyzed by ATC (Liege, Belgium).

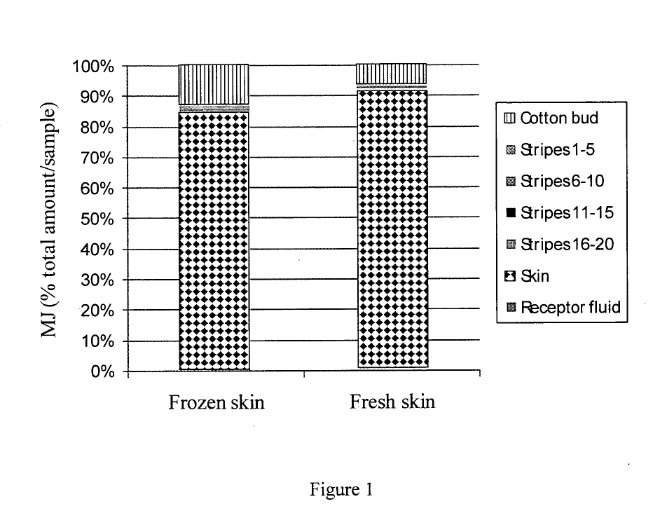

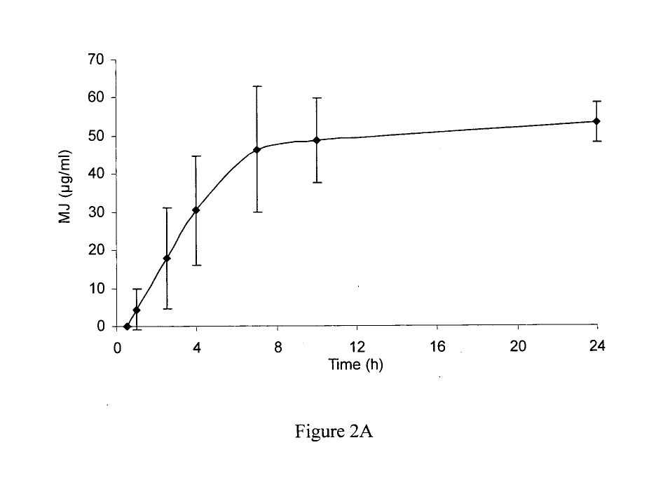

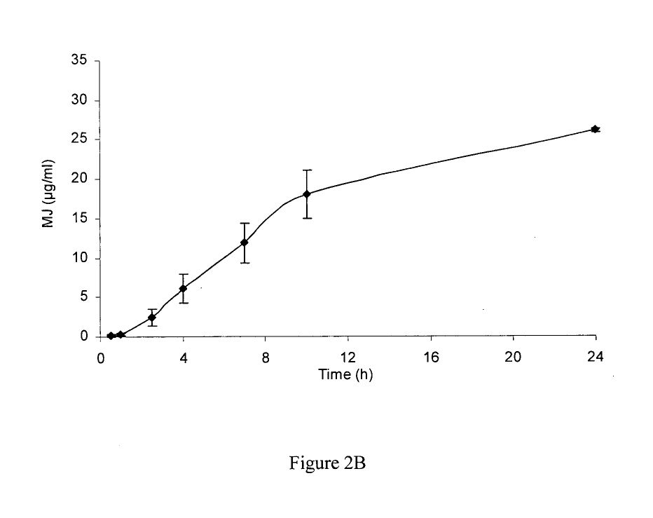

Two separate studies using cryo-preserved skin and fresh skin were conducted to test the percutaneous penetration of MJ. Upon application of MJ on skin patches, local intra-skin concentrations in the range of 1M were reached with no compound degradation. 24 hours after a single application, 80-90% of recovered MJ was found in the basal layer of the skin sample, corresponding mainly to epidermis and the upper part of the dermis. Approximately 0.3-1.2% was retrieved in the receptor fluid (FIG. 1). In the receptor fluid, MJ was detectable as early as one hour after application, with a plateau being reached after 8 to 12 hours characterizing a rapid skin penetration of the compound (FIGS. 2a and b).

Thus, it is clearly shown that MJ is easily absorbed in the skin and accumulates in the epidermis. These permeation studies on human skin indicate that compound degradation does not occur upon topical application of MJ thus enabling retention of very high concentration of MJ in the epidermis within the range of 1 mole/L.

### Example 2

### Toxicity of Jasmonate Ester Derivatives in Human Skin—Skin Irritation Assay

Acute irritation is a local, reversible inflammatory response of normal living skin to direct injury caused by the application of an irritant substance.

In order to test the toxicity of jasmonate ester derivatives in human skin, skin irritation assay using a reconstituted three-dimensional human epidermis model (EPISKIN-Standard Model™; conducted at BSL-Bioservices, Planegg, Germany) was performed. This skin model uses normal (non-cancerous), adult human-derived epidermal keratinocytes which have been cultured to form a multilayered, highly differentiated model of human epidermis with a functional stratum corneum.

In particular, MJ was applied topically to the EPISKIN-SM™ tissue for 15 minutes followed by a 42 hours post-incubation period and immediate determination of cytotoxic effects via MTT reduction assay. Irritant potential of the compound was assessed from the relative mean tissue viabilities obtained compared to the corresponding negative control tissues concurrently treated with Aqua Dest (distilled water). The release of Interleukin-1α (IL-1α) into the tissue culture medium was determined to confirm the obtained results.

MJ showed no irritant effects providing a mean relative viability >50% and IL-1α release ≦60 pg/ml (Table 1). MJ is thus classified as “non-irritant” according to the ECVAM SIVS recommendations.

TABLE 1
Skin irritation assay

| | Negative | | Positive |
|---|---|---|---|
| | control | | control 5% SDS |
| | Aqua Dest | MJ | solution |
| Mean relative tissue via- | 100 ± 6.7 | 106.7 ± 5.3 | 3.3 ± 0.6 |
| bility (%) (MTT reduction) | | | |
| IL-1α release (pg/ml) | 12.2 ± 7.9 | 28.3 ± 6.9 | 236.7 ± 23.0 |

### Example 3

### Toxicity of Jasmonate Ester Derivatives in Human Skin—Skin Corrosion Assay

Skin corrosion refers to the production of irreversible tissue damage in the skin following the application of a test material.

In order to test the toxicity of jasmonate ester derivatives in human skin, skin corrosion assay using a reconstituted three-dimensional human epidermis model (EpiDerm™ skin model from MatTek, conducted at BSL-Bioservices, Planegg, Germany) was performed. This skin model uses normal (non-cancerous), human-derived epidermal keratinocytes which have been cultured to form a multilayered, highly differentiated model of human epidermis with functional skin layers (basal, spinous, granular and cornified) analogous to those found in vivo.

In particular, MJ was applied topically to the EpiDerm™ tissue and incubated for 3 and 60 minutes at each time. After the incubation period, tissue viability was assessed via MTT reduction assay. The corrosive potential of MJ was assessed from the relative mean tissue viabilities obtained after 3 and 60 minutes compared to the corresponding negative control tissues concurrently treated with Aqua Dest (distilled water).

Using the EpiDerm™ skin model no corrosive effects were seen providing a mean relative tissue viability ≧50% after 3 min treatment and ≧15% after 60 min treatment (Table 2). MJ is thus classified as “non-corrosive” according to the OECD Guideline and the EC Commission Regulation.

TABLE 2
Skin corrosion assay

| | Negative | | Positive |
|---|---|---|---|
| | control | | control |
| | Aqua Dest | MJ | 8N KOH |
| Mean relative tissue viability (%) | 100 ± 10.6 | 106 ± 2.4 | 26 ± 5.3 |
| (MTT reduction) after 3 minutes | | | |
| Mean relative tissue viability (%) | 100 ± 2.4 | 94 ± 2.7 | 14 ± 3.7 |
| (MTT reduction) after 60 minutes | | | |

### Example 4

### Populations Recruited for Clinical Studies of Actinic Keratosis

### Inclusion Criteria:

1. Male or Female at least 18 years of age having one actinic keratosis lesion on the shoulders, chest, back or arms.
2. The longest diameter of the selected lesion is between 3 mm and 15 mm.
3. Screening laboratory values within the references ranges (as defined by the laboratory) or alternatively the values are “out of range” with acceptable variations.
4. Ability to follow study instructions, to complete all study requirements and having signed a written consent including a consent for photographs of the selected lesion to be taken and used as part of the study data package.

### Exclusion Criteria:

1. Females of child bearing potential.
2. Hypertrophic actinic keratoses.
3. Wherein the location of the selected actinic keratoses is:
   (i) within 5 cm of a scar.
   (ii) within 5 cm of any actinic keratosis lesion which is not selected for treatment.
   (iii) within 5 cm of an incompletely healed wound.
   (iv) on the breast.
   (v) within 5 cm of an area previously treated with surgical excision.
4. Presence of suspected basal cell carcinoma or squamous cell carcinoma within 5 cm of the selected treatment area.
5. Presence of known or suspected metastatic disease.
6. History or evidence of skin conditions other than actinic keratosis which would interfere with the evaluation of the study medication (e.g. eczema, unstable psoriasis, xeroderma pigmentosa).
7. A cosmetic or therapeutic procedure (e.g. liquid nitrogen, curettage, dermabrasion, medium or deep chemical peeling, laser resurfacing) located within 10 cm of the actinic keratosis lesion selected for treatment in the three months preceding the study, or anticipated treatment within 10 cm of the selected lesion during the study.
8. A cosmetic or therapeutic procedure located anywhere on the body in the four weeks preceding the study.
9. Treatment with 5-fluorouracil, imiquimod, diclofenac, masoprocol, or photodynamic therapy for lesions located within 10 cm of the actinic keratosis lesion selected for treatment in the three preceding the study.
10. Treatment with 5-fluorouracil, imiquimod, masoprocol, or photodynamic therapy for lesion located anywhere on the body in the four weeks preceding the study.
11. Previous treatment with other immunomodulators (e.g. vinblastine, podophyllin, colhamin, camptothecin), cytotoxic drugs (e.g. cyclophosphamide, azathioprine, chlorambucil, nitrogen mustard, methotrexate), or interferon/interferon inducers (other than imiquimod) in the four weeks preceding the study.
12. Previous treatment with psoralen plus UVA or use of UVB therapy on the six months preceding the study.
13. Patients who are excessively exposed to ultraviolet light (e.g. sunlight, tanning beds) during the study.
14. Use of medications that suppress the immune system (e.g. cyclosporine, prednisone, methotrexate, alefacept, infliximab) in the four weeks preceding the study.
15. Use of topical retinoids or light chemical peeling located within 10 cm of the actinic keratosis lesion selected for treatment in the four weeks preceding the study.
16. Use of systemic retinoids (e.g. isotretinoin, acitretin, bexarotene) in the six months preceding the study.
17. Use of acid containing products (e.g. salicylic acids or fruit acids such as α- and β-hydroxy acids and glycolic acids) located within 10 cm of the actinic keratosis lesion selected for treatment in the four weeks preceding the study.
18. Anticipated need to use acid containing products (e.g. salicylic acids or fruit acids, such as α- and β-hydroxy acids and glycolic acids) on the treatment area during the study.
19. Concurrent disease that suppresses the immune system (e.g. HIV).
20. Uncontrolled systemic disease (e.g. uncontrolled hypertension).
21. Anticipated need for surgery or hospitalization during the study.
22. Current evidence of chronic alcohol or drug abuse.
23. Current enrollment in an investigational drug or device study or participation in such a study in the 30 days preceding the study.

### Example 5

### Efficacy of Jasmonate Ester Derivatives in Treating Actinic Keratosis

In order to test the efficacy of jasmonate ester derivatives in treating actinic keratosis, 20 adult individuals having four or more clinically diagnosed actinic keratosis lesions of an approximate size of 25 cm² on sun exposed areas, in the shoulders, chest, back or arms are tested with a pure solution of methyl jasmonate. A total of 10 individuals are administered with a placebo as control population. Individuals who are excluded from the study are those who received any of the following treatments in the 30 days preceding the study: psoralen plus UVA therapy; UVB therapy; laser abrasion; dermabrasion or chemical peel. Throughout the study and in the 4 weeks preceding it, the following treatments are not allowed: topical retinoids, 5-fluorouracil, cryodestruction, chemodestruction, surgical excision, photodynamic therapy, curettage, interferon/interferon inducers, cytotoxic drugs, drugs with major organ toxicity, immunomodulators, immunosuppressive therapies, oral corticosteroids, or topical steroids anywhere on the treatment areas.

Treatment is performed by a daily administration of MJ or Placebo for 16 consecutive weeks, followed by a post-study period of 8 weeks. Approximately 10 μl of MJ or Placebo is applied topically over the lesion area.

The monitoring is performed as follows:
1. Initiation of administration (t=0): blood sampling is performed for the determination of pharmacokinetic parameters after the administration of the first dose.
2. t=3 days: haematological and biochemical assessments are performed for the evaluation of treatment tolerance.
3. t=4 weeks.
4. t=8 weeks.
5. t=16 weeks: end of treatment.
6. t=24 weeks: end of study.

Patients who discontinue the treatment period are asked to return for an assessment 8 weeks after their last dose treatment.

Efficacy, adverse events evaluation and photography are performed on each visit. Efficacy is evaluated by clinical counting and recording of the number of actinic keratosis present in the lesion area. The primary efficacy variable is the complete clearance rate, defined as the proportion of participants that have a total count of 0 clinically visible actinic keratosis lesions in the area that has been treated for a total of 8 weeks. The secondary efficacy variable is the partial clearance rate, defined as the proportion of participants that have at least a 75% reduction in the number of actinic keratosis lesions in the area that has been treated for a total of 8 weeks, in comparison to the initial number of actinic keratosis lesions.

Phase II double blind placebo study is then performed in order to assess the safety and efficacy of MJ in treating actinic keratosis. Safety of MJ in actinic keratosis patients is assessed for the administration of 10 μl pure solution of MJ applied topically over a 25 cm² treatment area surrounding a target lesion. The administration regimen is once daily for 16 consecutive weeks. Efficacy is assessed at 4 weeks, 8 weeks, 16 weeks and additional 8-weeks post-treatment.

### Example 6

### Tolerated Regimen and Safety of Jasmonate Ester Derivatives in Treating Actinic Keratosis

In order to test the tolerated regimen and safety of jasmonate ester derivatives in treating actinic keratosis, MJ is administered (10 μl gel applied topically over a 25 cm² treatment area surrounding a target lesion) once daily (hereinafter regimen A) or alternatively on odd days for 57 consecutive days (hereinafter regimen B) to patients with actinic keratosis (AK). Three patients are entered initially at each regimen to prevent the a regime limiting toxicity which is defined as ‘severe’ local skin reactions which appear either prior to treatment on even days (following treatment on Day 1) or observed on Day 8 (following the end of the treatment). If no RLT and/or systemic toxicity are observed, the patients who have not been treated are split into two groups; each group is subjected to a different administration regimen (A or B). Systemic absorption and local tolerability are assessed on Day 1 and Day 8. The clinical efficiency of MJ at both regimes for 8 consecutive weeks is assessed through measurements of hematologic and biochemical parameters along with cosmetic assessment that are undertaken at the screening visit on Day 0, Days 8 and Day 57. Adverse events are assessed at every study visit. Clinical response to treatment is assessed on Days 8 and Day 57 and additionally 8 weeks after the last dose treatment.

Phase II study is then performed in order to assess optimal regimen of MJ in treating actinic keratosis. Optimal tolerated regime of MJ in actinic keratosis patients is assessed when administered once daily or alternatively only on odd days for 56 consecutive days of 10 μl pure solution of MJ applied topically over a 25 cm² treatment area surrounding a target lesion. Efficacy is assessed at 8 and 57 days after application of MJ at the optimal tolerated regime in the expanded cohort.

### Example 7

### Avian Model of Benign Hyperproliferative Skin Disorders

In order to test the effect of jasmonate ester derivatives on benign hyperproliferative disorders of the skin, intact mammalian skin explants grafted on the chorioallantoic membrane (CAM) of a fertilized avian are used as an animal model for actinic keratoses or psoriasis. This model is disclosed in PCT international application publication number WO 2006/001021, the contents of which are incorporated by reference herein in their entirety as if fully set forth herein.

Briefly, freshly-laid fertile chicken eggs are stored at 15° C. until required. The eggs are then warmed for one hour to room temperature, followed by vertical (with the point down) incubation in a humid atmosphere at 37° C. for 5-10 days before use. On the third day of incubation, the eggs are turned upside down, and a small hole is made in the sharp side of the egg after cleaning with a tissue impregnated with 70% ethanol. This creates an artificial air sac so that the CAM can be accessed later on without causing bleeding.

Human actinic keratosis or psoriatic lesions are removed from patients. The skin is pinned out in a petri dish with a rubber bottom, and cut into rectangular/square pieces approximately 5-10 mm on each side using scissors, scalpels or dermatological punches. The skin is stored at 15° C. in PBS⁺⁺ until grafting. Approximately 3-6 mm diameter punch biopsies of full thickness human skin are cultured at an air-liquid interface on a plastic mesh insert in 12-well culture plates in MEM medium (high Ca⁺⁺), 10% fetal calf serum and antibiotics.

Eggs with an artificial air sac are opened with iris scissors in a sterile hood. The ectodermal surface of the CAM is abraded by touching it briefly with a sterile piece of lens tissue to improve the adherence of the graft. Each piece of skin is then gently placed on the CAM and stretched out. The eggs are then sealed with adhesive tape, and returned to the incubator.

After grafting of the punch biopsies of human actinic keratosis or psoriatic lesions, the lesions are allowed to incubate for 2 days in order to allow for the skin to incorporate, and then the adhesive tape sealing of the samples is reopened. Methyl jasmonate is topically applied to different skin samples at different concentrations using a small plastic ring cut from a pipette tip. The samples are then resealed and returned to the incubator for an addition three days, whereupon they undergo routine histological and immunochemical analysis using Abs for the proliferation marker PCNA. PCNA⁺ cells are counted in several sections, and the length of the epidermis in the section measured. Analysis includes skin fixation in 4% paraformaldehyde or Bouin's fluid, followed by paraffin embedding, cutting into 6 μm sections and staining either with hematoxylin and eosin (H&E) or immunostaining with skin specific keratin antibodies (K10, K14), and counterstaining with Hoechst nuclear staining.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1-32. (canceled)

33. A method of treating keratosis, comprising the step of administering to a subject in need thereof a pharmaceutical composition comprising methyl jasmonate.

34. The method according to claim 33, wherein the composition is formulated for topical administration.

35. The method according to claim 33, wherein the keratosis is selected from at least one of actinic keratosis, hypertrophic actinic keratosis, Bowenoid actinic keratosis, arsenical keratosis, hydrocarbon keratosis, thermal keratosis, radiation keratosis, chronic scar keratosis, viral keratosis, actinic cheilitis, Bowen's disease, erythroplaquia of queyrat, oral erythroplaquia, leukoplakia or intraepidermal epithelialoma.

36. The method according to claim 33, wherein the keratosis is actinic keratosis.

37. A method of treating a benign hyperproliferative skin disorder in a subject in need thereof, comprising administering to the subject an effective amount of a composition comprising at least one jasmonate ester derivative, wherein the benign hyperproliferative skin disorder is not psoriasis and the jasmonate ester derivative is not methyl dihydro jasmonate.

38. The method according to claim 37, wherein the jasmonate ester derivative is methyl jasmonate.

39. The method according to claim 37, wherein the jasmonate ester derivative is represented by the structure of any of formulae A, B, C, I, II, III, IV, V, VI or VII.

40. The method according to claim 37, wherein the composition is formulated for topical administration.

41. The method according to claim 40, wherein the benign hyperproliferative skin disorder is selected from the group consisting of keratoses, actinic keratosis, common warts, keratoacanthoma, seborrhoic keratosis, seborrhea and ichthyosis.

42. The method according to claim 41, wherein the keratoses are selected from the group consisting of actinic keratosis, hypertrophic actinic keratosis, Bowenoid actinic keratosis, arsenical keratosis, hydrocarbon keratosis, thermal keratosis, radiation keratosis, chronic scar keratosis, viral keratosis, actinic cheilitis, Bowen's disease, erythroplaquia of queyrat, oral erythroplaquia, leukoplakia or intraepidermal epithelialoma.

43. The method according to claim 41, wherein the skin disorder is actinic keratosis.

44. An assay for determining the therapeutic effect of a jasmonate ester derivative in benign hyperproliferative skin disorders, comprising
(a) introducing a viable mammalian skin explant obtained from skin having a benign hyperproliferative lesion, into a mammalian-avian chimeric model system comprising a fertilized avian egg within an egg shell, wherein a portion of the egg shell is removed creating an aperture, wherein the skin explant is in contact with the chorioallantoic membrane (CAM) of the fertilized avian egg such that vasculature extends from said fertilized avian egg to said explant;
b) incubating said fertilized avian egg for a period of time to allow engraftment;
c) contacting at least a portion of said explant with at least one jasmonate ester derivative; and
d) examining said explant for a beneficial effect of the jasmonate ester derivative on the skin pathology.

45. The assay according to claim 44, wherein the benign hyperproliferative skin disorder is selected from the group consisting of keratoses, actinic keratosis, common warts, keratoacanthoma, seborrhoic keratosis, seborrhea and ichthyosis.

46. The assay of claim 44 wherein exposing at least a portion of said explant to the at least one jasmonate ester derivative occurs prior to step (a).

47. The assay of claim 44 wherein the at least one jasmonate ester derivative is contacted with the explant by topical administration, subcutaneous administration, injection into the explant, injection into the explant vasculature or injection into the fertilized avian egg vasculature.

48. The assay of claim 44 wherein the at least a portion of said explant-egg system examined in step (d) is selected from the group consisting of: at least a portion of the engrafted explant; at least a portion of the fertilized egg; at least a portion of a hematopoeitic organ of the avian embryo selected from the group consisting of spleen, bone marrow and liver; a sample of blood extracted from the explant vasculature; a sample of blood extracted from the fertilized egg vasculature; and a sample of waste extracted from the allantois of the fertilized egg.

49. The assay of claim 44 wherein at least a portion of said explant is examined using histological techniques, immunocytochemical techniques, biochemical techniques, molecular techniques, flow cytometry and polymerase chain reaction (PCR), techniques forestimating cell proliferation rate, techniques for assessing connective tissue synthesis, measurements of tissue elasticity, techniques for assessing blood vessel formation, and methods for determining epidermal differentiation, skin inflammation or fat deposition.

50. The assay of claim 44 wherein said explant is obtained from human skin.

51. The assay of claim 44 wherein the jasmonate ester derivative is methyl jasmonate.

52. The assay of claim 44, wherein the jasmonate ester derivative is represented by the structure of any of formulae A, B, C, I, II, III, IV, V, VI or VII.
